(19) United States
(12) Patent Application Publication
(10) Pub. No.: US 2026/0202353 A1
(43) Pub. Date: Jul. 16, 2026

(54) INTEGRATED REAL-TIME RF ANALYSIS SYSTEM AND METHOD FOR FOOD SAFETY ANALYSIS

(71) Applicant: Know Labs, Inc., Seattle, WA (US)

(72) Inventor: John Cronin, Jericho, VT (US)

(21) Appl. No.: 19/384,894

(22) Filed: Nov. 10, 2025

Related U.S. Application Data
(60) Provisional application No. 63/718,656, filed on Nov. 10, 2024.

(51) Int. Cl. G01N 22/00; G01N 33/02

(52) U.S. Cl. CPC G01N 22/00; G01N 33/02

(57) ABSTRACT

The present disclosure provides an integrated real-time RF analysis system and method for food safety analysis in which the sensor module collects data from the RF sensor and correlates the RF data with ground truth data to determine the levels of a plurality of parameters of the food, the integration module compares the parameters of the food to an integration database to determine if there is a corresponding notification, extracts the corresponding notification and displays the notification to inform a user of the safety of the food.

### FIELD OF THE DISCLOSURE

The present disclosure is generally related to an integrated real-time RF analysis system and method for food safety analysis.

### BACKGROUND

Currently, certain food analyzers may have limitations in detecting low levels of contaminants or spoilage indicators. In some cases, the sensitivity of the sensors may not be sufficient to identify trace amounts of harmful substances or early stages of spoilage. Ensuring the specificity of the analysis is crucial. Some analyzers may encounter challenges distinguishing between different types of contaminants or spoilage markers. For accurate results, the device must be capable of identifying specific pathogens or spoilage-related compounds. Also, some analyzers require complex or time-consuming sample preparation procedures, which can be impractical for routine use. Simplifying sample preparation without compromising accuracy remains a goal for improving food analyzers. The speed of analysis can be a limitation, especially in high-throughput food processing facilities or during inspections. Faster analysis times would enable more efficient quality control and safety assessment. Lastly, many analyzers excel at detecting specific contaminants or spoilage markers individually, but challenges arise when trying to analyze multiple contaminants simultaneously. Multiplexing capabilities are essential for comprehensive safety assessments. Interpreting the data generated by food analyzers can be challenging, particularly when dealing with vast amounts of information. Efficient data processing and analysis tools are necessary to extract meaningful insights from the collected data. Thus, there is a need in the prior art for an integrated real-time RF analysis system and method for food safety analysis. There are limitations or challenges with real-time food spoilage detection devices, such as sensor drift over time can affect accuracy, not presenting a full picture of spoilage, some algorithms require more development, some sensors are affected by humidity and temperatures resulting in recalibration, etc.

### DESCRIPTION OF THE DRAWINGS

FIG. 1: Illustrates an example of an integrated real-time RF analysis system for food safety analysis, according to an embodiment.

FIG. 2: Illustrates an example operation of a Base Module, according to an embodiment.

FIG. 3: Illustrates an example operation of a Sensor Module, according to an embodiment.

FIG. 4: Illustrates an example operation of an Integration Module, according to an embodiment.

FIG. 5: Illustrates an example of an Integration Database, according to an embodiment.

FIG. 6: Illustrates an example of a Sensor Database, according to an embodiment.

### DETAILED DESCRIPTION

Embodiments of the present disclosure will be described more fully hereinafter with reference to the accompanying drawings in which like numerals represent like elements throughout the several figures, and in which example embodiments are shown. Embodiments of the claims may, however, be embodied in many different forms and should not be construed as limited to the embodiments set forth herein. The examples set forth herein are non-limiting examples and are merely examples among other possible examples.

U.S. Pat. Nos. 10,548,503, 11,063,373, 11,058,331, 11,033,208, 11,284,819, 11,284,820, 10,548,503, 11,234,619, 11,031,970, 11,223,383, 11,058,317, 11,193,923, 11,234,618, 11,389,091, U.S. 2021/0259571, U.S. 2022/0077918, U.S. 2022/0071527, U.S. 2022/0074870, U.S. 2022/0151553, are each individually incorporated herein by reference in its entirety.

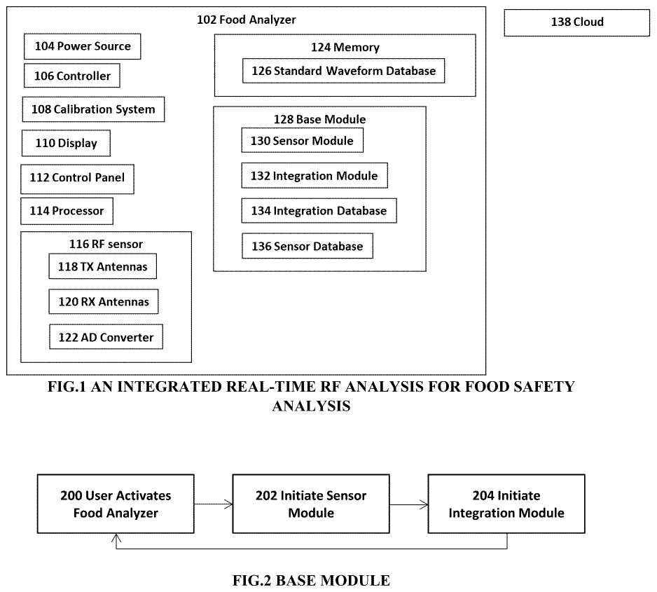

FIG. 1 illustrates a system providing an integrated real-time RF analysis for food safety analysis. This system may be comprised of a food analyzer 102, which may be an instrument designed to assess and determine the safety and quality of food products. The food analyzer 102 may include an RF sensor 116 that collects RF data and, through an integration module 132, determines the levels of various parameters, including pH levels, bacteria levels, volatile organic compounds, enzyme levels, oxidation levels, etc., to determine if the food is safe to consume. The food analyzer 102 may include a power source 104, controller 106, calibration system 108, display 110, control panel 112, processor 114, and memory 124.

Further, embodiments may include a power source 104, which may be an electronic component capable of supplying electrical energy to a device. A power source 104 can be a battery, a fuel cell, a power supply unit, or any other type of device that generates or stores electrical energy. The power source 104 can provide either direct current (DC) or alternating current (AC) to the device and may be rechargeable or non-rechargeable. In some embodiments, the power source 104 may be designed to provide a specific voltage or current output, or it can be regulated by a voltage regulator or current regulator. Additionally, the power source 104 may be controlled by a microcontroller or other electronic circuitry to manage power consumption and optimize performance. The power source 104 may also include monitoring circuitry to detect its own charge level and communicate that information to the device or an external source. In certain embodiments, the power source 104 can include a charging circuit that allows the device to be charged by an external power source, such as a wall outlet or a USB port.

Further, embodiments may include a controller 106, which may be responsible for controlling and managing the operation of the food analyzer 102. The controller 106 may include a set of algorithms, rules, and logic circuits that are used to process input signals and generate output signals to control the behavior of the food analyzer 102. In some embodiments, the controller 106 may be a microcontroller, programmable logic controller (PLC), or digital signal processor (DSP) and may be implemented as software components running on general-purpose computers or embedded systems.

Further, embodiments may include a calibration system 108, which may be a set of processes and procedures designed to ensure the accuracy and precision of the various measurement and control components within the food analyzer 102. The calibration system 108 ensures that the displayed values and measured parameters are consistent and accurate.

Further, embodiments may include a display 110, which may be a hardware component that provides visual output for a computer system, also known as a monitor or screen. The display 110 may be comprised of a panel, controller, and other subsystems. The display 110 may be the physical component that emits light and creates the image on a screen. The display 110 may consist of a thin film transistor (TFT) layer and a liquid crystal layer, which work together to control the flow of light through the panel and create an image. The display 110 may include components for decoding video signals and scaling images to the appropriate size and resolution to create the final image. The display 110 may include backlighting systems which provide uniform illumination across the panel and enhance the brightness and contrast of the displayed image. The display 110 may include touch-sensitive screens, which enable users to interact with the system using gestures and touch inputs. A display 110 may also include advanced color management systems, which provide accurate color reproduction and enable users to calibrate the display 110 to match their preferences and requirements.

Further, embodiments may include a control panel 112, which may be a user interface that allows the user to set, adjust, and monitor various parameters and functions of the food analyzer 102. The control panel 112 may serve as the primary means of communication between the user and the food analyzer 102 internal systems and software.

Further, embodiments may include a processor 114, also known as a central processing unit (CPU), which may facilitate the operation of the food analyzer 102 according to the instructions stored in the memory 124. The processor 114 may include suitable logic, circuitry, interfaces, and/or code that may be configured to execute a set of instructions stored in the memory 124. The processor 114 may be a hardware component that performs arithmetic, logic, and control operations on data. The processor 114 may be comprised of the arithmetic logic unit, control unit, memory subsystems, and other subsystems. The processor 114 may be responsible for performing arithmetic and logical operations on data. The processor 114 may include components for addition, subtraction, multiplication, and division and logical operations such as AND, OR, and NOT. The processor 114 may be responsible for fetching instructions from memory 124, decoding them, and executing them. The processor 114 may manage the flow of data between different components of the system as a whole, ensuring that operations are performed in the correct order, and that data is transferred efficiently. The processor 114 may provide fast access to frequently used data and instructions. The processor 114 may include components such as caches, registers, and pipelines, which are designed to minimize the time required to access and manipulate data. The processor 114 may include various other components and subsystems, such as instruction set architecture (ISA), which may define the set of instructions that the processor 114 can execute. The processor 114 may specify the format of instructions and data, the addressing modes used to access memory 124 and I/O devices, and the interrupt and exception handling mechanisms used to manage errors and other events. The processor 114 may include advanced instruction execution capabilities, support for virtualization and parallel processing, and power management mechanisms that reduce energy consumption and heat dissipation.

Further, embodiments may include an RF sensor 116, which may contain a plurality of TX antennas 118, a plurality of RX antennas 120, and an AD converter 122, etc.

Further, embodiments may include a plurality of TX antennas 118 which may be integrated into the circuitry arrangement. The one or more TX antennas 118 may be configured to transmit the Activated RF range signals at a predefined frequency. In one embodiment, the predefined frequency may correspond to a range suitable for food. For example, the one or more TX antennas 118 transmit Activated RF range signals at a predefined range, such as 500 MHz to 300 GHz.

Further, embodiments may include a plurality of RX antennas 120 which may be integrated into the circuitry arrangement. The one or more RX antennas 120 may be configured to receive the responded portion of the Activated RF range signals. In one embodiment, the Activated RF range signals may be transmitted into the food, and electromagnetic energy may be responded from many elements of the food. It can be noted that effective monitoring of the parameter levels is facilitated by an electrical response of parameters against the transmitted Activated RF range signals. Further, the electromagnetic energy responded from the food may be received by the one or more RX antennas 120.

Further, embodiments may include an AD converter 122, which may be coupled to the one or more RX antennas 120. The one or more RX antennas 120 may be configured to receive the responded Activated RF range signals. The AD converter 122 may be configured to convert the Activated RF range signals from an analog signal into a digital processor readable format.

Further, embodiments may include a memory 124, which may be configured to store the transmitted Activated RF range signals by the one or more TX antennas 118 and receive a responded portion of the transmitted Activated RF range signals from the one or more RX antennas 120. Further, the memory 124 may also store the converted digital processor readable format by the AD converter 122. The memory 124 may store data collected by the food analyzer 102, such as sensor data, analysis of data, etc. In one embodiment, the memory 124 may include suitable logic, circuitry, and/or interfaces that may be configured to store a machine code and/or a computer program with at least one code section executable by the processor 114. Examples of implementation of the memory 124 may include, but are not limited to, Random Access Memory (RAM), Read Only Memory (ROM), Hard Disk Drive (HDD), and/or a Secure Digital (SD) card.

Further, embodiments may include a standard waveform database 126, which may contain standard waveforms for known patterns. These may be raw or converted RF sensor 116 readings from different types of food with known parameters. For example, the standard waveform database 126 may include raw or converted RF sensor 116 readings from food sample parameters or an average of multiple food samples. This data can be compared to readings from a food with unknown parameters to determine if the waveforms from that food match any known standard waveforms. The standard waveform database 126 may include transmit signals and response signals for specific types of food, a specific type or parameter, and the parameter level, etc., which may enable the sensor module 130 to send a plurality of transmit signals to determine the type of food and the parameter levels for a plurality of parameters. For example, the user may activate the food analyzer 102, and the first transmit signal may be 500 MHz, and the response signal may be 425 MHz, and the transmit signal and response signal may correlate to the type of food being beef but not correlate to the food being pork, chicken, fish, etc., allowing the sensor module 130 to determine the food is beef. The sensor module 130 may then send a plurality of transmit signals to the beef to determine the various parameter levels of the beef to determine if the parameter levels are in a safe or unsafe range once compared to the integration database 134 in the process described in the integration module 132.

Further, embodiments may include a base module 128, which begins with the user activating the food analyzer 102, initiating the sensor module 130, and initiating the integration module 132.

Further, embodiments may include a sensor module 130, which begins by being initiated by the base module 128. In some embodiments, the sensor module 130 may be initiated through the user activating the food analyzer 102 by selecting an option on the control panel 112. In some embodiments, the sensor module 130 may be initiated a plurality of times or instances to collect data on multiple parameters of the food being analyzed. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 sends the RF transmit signal to the TX antennas 118. The one or more TX antennas 118 may be configured to transmit the Activated RF range signals at a predefined frequency. In one embodiment, the predefined frequency may correspond to a range suitable for food. For example, the one or more TX antennas 118 transmit Activated RF range signals at a range of 500 MHz to 300 GHz. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 stores the RF transmit signal to memory 124. The sensor module 130 stores the transmitted signal to memory 124, such as Activated RF range signals at a range of 500 MHz to 300 GHz. The sensor module 130 receives the RF signal from the RX antennas 120. The one or more RX antennas 120 may be configured to receive the responded portion of the Activated RF range signals. In one embodiment, the Activated RF range signals may be transmitted into the food, and electromagnetic energy may be responded from the food. It can be noted that effective monitoring of the parameter levels is facilitated by an electrical response of parameters against the transmitted Activated RF range signals. Further, the electromagnetic energy responded from the food may be received by the one or more RX antennas 120. The sensor module 130 converts to digital using the AD converter 122. For example, the AD converter 122 may be configured to convert the Activated RF range signals from an analog signal into a digital processor readable format. The sensor module 130 stores the RX converted signal data in memory 124. The sensor module 130 stores the received signal from the RX antennas 120 that has been converted to a digital processor readable format in memory 124. The sensor module 130 correlates the RF signals with ground truth data to determine the parameter levels. The sensor module 130 may be configured to execute an AI correlation between the real-time ground truth data and the RX-converted data. In one embodiment, the AI correlation between the real-time ground truth data and the RX-converted data is executed to determine whether the RX-converted data corresponds to the real-time ground truth data. The ground truth data may be determined by another sensor that identifies the parameter levels when a waveform was transmitted and received. Machine learning processes may be performed to identify specific parameter waveforms, in which complex responded signals from stepped frequencies transmit signals that may be related to parameter levels. The memory 124 is used in real-time to compare received waveforms from the RX antennas 120 to a standard waveform database 126 stored in memory 124 to identify the parameter level for the received waveform from the RX antennas 120. In one embodiment, the standard waveform database 126 may be configured to store the filtered RF signal received from the one or more RX antennas 120 of the food analyzer 102. The standard waveform database 126 may store the signal waveforms for the TX antennas 118 and the received signal waveforms for the RX antennas 120. The database may include the parameter levels with the corresponding signal waveform, received waveform, and the TX antennas 118 and RX antennas 120 that were used. The standard waveform database 126 may include transmit signals and response signals for specific types of food, a specific type or parameter, and the parameter level, etc., which may enable the sensor module 130 to send a plurality of transmit signals to determine the type of food and the parameter levels for a plurality of parameters. For example, the user may activate the food analyzer 102, and the first transmit signal may be 500 MHz, and the response signal may be 425 MHz, and the transmit signal and response signal may correlate to the type of food being beef but not correlate to the food being pork, chicken, fish, etc., allowing the sensor module 130 to determine the food is beef. The sensor module 130 may then send a plurality of transmit signals into the beef to determine the various parameter levels of the beef to determine if the parameter levels are in a safe or unsafe range once compared to the integration database 134 in the process described in the integration module 132. The sensor module 130 stores the parameter levels in the sensor database 136. The sensor module 130 may store the type of food detected and the parameter levels, such as the food's pH levels, bacteria levels, levels of volatile organic compounds, enzyme levels, oxidation levels, etc. in the sensor database 136. In some embodiments, the sensor module 130 may collect parameter levels of the food by sending, receiving, converting, and then correlating the data for a first parameter and then a second parameter separately, in parallel, etc. The sensor module 130 returns to the base module 128.

Further, embodiments may include an integration module 132 which begins by being initiated by the base module 128. In some embodiments, the integration module 132 may be initiated when a new data entry is stored in the sensor database 136 to determine if a corresponding notification is stored in the integration database 134. The integration module 132 extracts the data stored in the sensor database 136. The integration module 132 extracts the data, such as the detected food type, the parameter being measured, and the parameter level. The integration module 132 compares the extracted data from the sensor database 136 to the integration database 134. The integration database 134 may be a preloaded or previously created database that may contain a set of rules that, if met, a corresponding notification may be extracted and displayed on the display 110 to notify the user of the health and safety status of the food. The integration database 134 may contain a list of ranges for a plurality of food parameters in which if the parameter levels fall in-between, above, or below the ranges, a corresponding notification may be displayed to the user. The integration database 134 may contain the type of food detected, the parameter that is being measured, the parameter range or threshold, and the corresponding notification. In some embodiments, the integration database 134 may contain safe ranges, unsafe ranges, ranges related to the time remaining until the food spoils, etc., such as pH levels, bacteria levels, volatile organic compounds, enzyme levels, oxidation, lipid content, protein content, myoglobin concentration, trimethylamine, total volatile basic nitrogen, free fatty acids, water content, residual antibiotics, and pesticides, etc., etc. Fresh meat has very low levels of bacteria, and as it spoils, bacteria like pseudomonas, lactobacillus, and yeast will rapidly multiply, and measuring these can indicate spoilage. Acceptable levels are below 105 colony-forming units per gram. Spoiling meat gives off chemicals like putrescine, cadaverine, and ammonia. These Volatile organic compounds levels increase dramatically as meat goes bad and may be measured to check freshness. Enzymes like catalase and galactosidase are produced during spoilage, and measuring these enzyme levels may help determine meat freshness. Rancidity from fat oxidation causes meat to spoil and may be measured by checking for substances like peroxides, aldehydes, and ketones. The integration module 132 determines if a corresponding notification is stored in the integration database 134. For example, if the food detected is beef or pork and the parameter is the pH level of the food, the parameter range may be 5.4-6.2 pH, and the corresponding notification may be that the beef or pork was detected and is safe to consume. If the food type detected is chicken and the parameter is the pH level of the food, the parameter range may be 5.5-6.5 pH, and the corresponding notification may be that chicken was detected and is safe to consume. Meat with pH levels within these safe ranges is considered suitable for consumption. However, if the pH level of meat falls outside these ranges, it may indicate potential safety concerns. If the food type detected is beef and the parameter is the pH level, and the beef's pH level is above 6.5, the corresponding notification may be that beef was detected and is not safe to consume. If the food type detected is chicken and the parameter is the pH level, and the chicken's pH level is below 5, the corresponding notification may be that chicken was detected and is not safe to consume. If the food type detected is pork and the parameter is the pH level, and the pork's pH level is above 6.5, the corresponding notification may be that pork was detected and is not safe to consume. A pH level higher than the safe range could indicate the presence of harmful bacteria or spoilage microorganisms and may lead to the development of undesirable flavors and textures in the meat. A pH level lower than the safe range might indicate excessive acid production due to prolonged storage, spoilage, or fermentation. Highly acidic meat may have an off-putting sour taste and may not be safe for consumption. If the food type detected is chicken and the parameter is Salmonella, the parameter range may be above 1 CFU, colony-forming unit, per gram with a corresponding notification of detected chicken, salmonella warning, do not consume. Salmonella should ideally be absent or at very low levels in meat products. The safe range is generally considered to be less than 1 CFU (colony-forming unit) per gram. Dangerous levels of Salmonella in meat would be anything above the acceptable safe range, such as greater than 1 CFU per gram. High levels of Salmonella pose a significant risk of causing foodborne illness. If the food type detected is beef and the parameter is Campylobacter, the parameter range may be above 100 CFU per gram with a corresponding notification of detected beef, campylobacter warning, do not consume. Campylobacter should ideally be absent or at very low levels in meat products, with a safe range generally considered to be less than 100 CFUs per gram. Dangerous levels of Campylobacter would be above the acceptable safe range, such as greater than 100 CFUs per gram, and high levels of Campylobacter can cause foodborne illness. If the food type detected is pork and the parameter is E. coli, the parameter range may be above 1 CFU per gram with a corresponding notification of detected pork, E. coli warning, do not consume. E. coli is naturally present in the intestines of animals and humans, but certain strains can be harmful, and the safe range for pathogenic E. coli in meat is typically less than 1 CFU per gram. Dangerous levels of pathogenic E. coli would be above the acceptable safe range, such as greater than 1 CFU per gram. High levels of pathogenic E. coli can lead to severe foodborne illnesses. If it is determined that there is a corresponding notification stored in the integration database 134, the integration module 132 extracts the notification from the integration database 134. The integration module 132 extracts the notification, such as beef was detected and is safe to consume, the chicken was detected and is safe to consume, pork was detected and is safe to consume, beef was detected and is not safe to consume, the chicken was detected and is not safe to consume, pork was detected and is not safe to consume, detected chicken, salmonella warning, do not consume, detected beef, campylobacter warning, do not consume, detected pork, E. coli warning, do not consume. The integration module 132 displays the extracted notification from the integration database 134 on the display 110. The integration module 132 displays the notification, such as beef was detected and is safe to consume, the chicken was detected and is safe to consume, pork was detected and is safe to consume, beef was detected and is not safe to consume, chicken was detected and is not safe to consume, pork was detected and is not safe to consume, detected chicken, salmonella warning, do not consume, detected beef, campylobacter warning, do not consume, detected pork, E. coli warning, do not consume. If it is determined that there is not a corresponding notification stored in the integration database 134 or after the notification is displayed, the integration module 132 returns to the base module 128.

Further, embodiments may include an integration database 134, which may be a preloaded or previously created database that may contain a set of rules that, if met, a corresponding notification may be extracted and displayed on the display 110 to notify the user of the health and safety status of the food. The integration database 134 may contain a list of ranges for a plurality of food parameters in which if the parameter levels fall in-between, above, or below the ranges, a corresponding notification may be displayed to the user. The integration database 134 may contain the type of food detected, the parameter that is being measured, the parameter range or threshold, and the corresponding notification. For example, if the food detected is beef or pork and the parameter is the pH level of the food, the parameter range may be 5.4-6.2 pH, and the corresponding notification may be that the beef or pork was detected and is safe to consume. If the food type detected is chicken and the parameter is the pH level of the food, the parameter range may be 5.5-6.5 pH, and the corresponding notification may be that chicken was detected and is safe to consume. Meat with pH levels within these safe ranges is considered suitable for consumption. However, if the pH level of meat falls outside these ranges, it may indicate potential safety concerns. If the food type detected is beef and the parameter is the pH level, and the beef's pH level is above 6.5, the corresponding notification may be that beef was detected and is not safe to consume. If the food type detected is chicken and the parameter is the pH level, and the chicken's pH level is below 5, the corresponding notification may be that chicken was detected and is not safe to consume. If the food type detected is pork and the parameter is the pH level, and the pork's pH level is above 6.5, the corresponding notification may be that pork was detected and is not safe to consume. A pH level higher than the safe range could indicate the presence of harmful bacteria or spoilage microorganisms and may lead to the development of undesirable flavors and textures in the meat. A pH lower than the safe range might indicate excessive acid production due to prolonged storage, spoilage, or fermentation. Highly acidic meat may have an off-putting sour taste and may not be safe for consumption. If the food type detected is chicken and the parameter is Salmonella, the parameter range may be above 1 CFU, colony-forming unit, per gram with a corresponding notification of detected chicken, salmonella warning, do not consume. Salmonella should ideally be absent or at very low levels in meat products. The safe range is generally considered to be less than 1 CFU (colony-forming unit) per gram. Dangerous levels of Salmonella in meat would be anything above the acceptable safe range, such as greater than 1 CFU per gram. High levels of Salmonella pose a significant risk of causing foodborne illness. If the food type detected is beef and the parameter is Campylobacter, the parameter range may be above 100 CFU per gram with a corresponding notification of detected beef, campylobacter warning, do not consume. Campylobacter should ideally be absent or at very low levels in meat products, with a safe range generally considered to be less than 100 CFUs per gram. Dangerous levels of Campylobacter would be above the acceptable safe range, such as greater than 100 CFUs per gram, and high levels of Campylobacter can cause foodborne illness. If the food type detected is pork and the parameter is E. coli, the parameter range may be above 1 CFU per gram with a corresponding notification of detected pork, E. coli warning, do not consume. E. coli is naturally present in the intestines of animals and humans, but certain strains can be harmful, and the safe range for pathogenic E. coli in meat is typically less than 1 CFU per gram. Dangerous levels of pathogenic E. coli would be above the acceptable safe range, such as greater than 1 CFU per gram. High levels of pathogenic E. coli can lead to severe foodborne illnesses. In some embodiments, the integration database 134 may contain safe ranges, unsafe ranges, ranges related to the time remaining until the food spoils, etc., such as pH levels, bacteria levels, volatile organic compounds, enzyme levels, oxidation, lipid content, protein content, myoglobin concentration, trimethylamine, total volatile basic nitrogen, free fatty acids, water content, residual antibiotics, and pesticides, etc., etc. Fresh meat has very low levels of bacteria, and as it spoils, bacteria like pseudomonas, lactobacillus, and yeast will rapidly multiply, and measuring these can indicate spoilage. Acceptable levels are below 105 colony-forming units per gram. Spoiling meat gives off chemicals like putrescine, cadaverine, and ammonia. These Volatile organic compounds levels increase dramatically as meat goes bad and may be measured to check freshness. Enzymes like catalase and galactosidase are produced during spoilage, and measuring these enzyme levels may help determine meat freshness. Rancidity from fat oxidation causes meat to spoil and may be measured by checking for substances like peroxides, aldehydes, and ketones.

Further, embodiments may include a sensor database 136, which may be created during the process described in the sensor module 130 in which the food type and the parameter levels are stored through the data collected by the RF sensor 116. The sensor database 136 may contain the detected food type, the parameter being measured, and the parameter level. The sensor database 136 may contain a plurality of parameter readings which are compared to the ranges and thresholds stored in the integration database 134 through the process described in the integration module 132 to determine if the food being analyzed is safe for consumption or has unsafe parameter levels.

Further, embodiments may include a cloud 138, which may be a network of remote servers that provide on-demand computing resources and services over the Internet. The cloud 138 may consist of a collection of servers, storage devices, and networking equipment. Users may access the cloud 138 through a variety of devices, such as computers, smartphones, and tablets, using internet connectivity. In some embodiments, the architecture of the cloud 138 may be based on a distributed computing model, with multiple servers working together to provide services to users.

FIG. 2 illustrates an example operation of the base module 128. The process begins with the user activating, at step 200, the food analyzer 102. In some embodiments, the user may input in the control panel 112 the type of food that is being analyzed. In some embodiments, the user may activate the food analyzer 102 by placing the food analyzer 102 against or near the food and selecting an option on the control panel 112 to collect the data to determine if the food is safe or spoiled. In some embodiments, the user may select the parameters the food analyzer 102 should check for, such as pH level, bacteria levels, levels of various volatile organic compounds, enzyme levels, oxidation levels, etc. The base module 128 initiates, at step 202, the sensor module 130. The sensor module 130 begins by being initiated by the base module 128. In some embodiments, the sensor module 130 may be initiated through the user activating the food analyzer 102 by selecting an option on the control panel 112. In some embodiments, the sensor module 130 may be initiated a plurality of times or instances to collect data on multiple parameters of the food being analyzed. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 sends the RF transmit signal to the TX antennas 118. The one or more TX antennas 118 may be configured to transmit the Activated RF range signals at a predefined frequency. In one embodiment, the predefined frequency may correspond to a range suitable for food. For example, the one or more TX antennas 118 transmit Activated RF range signals at a range of 500 MHz to 300 GHz. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 stores the RF transmit signal to memory 124. The sensor module 130 stores the transmitted signal to memory 124, such as Activated RF range signals at a range of 500 MHz to 300 GHz. The sensor module 130 receives the RF signal from the RX antennas 120. The one or more RX antennas 120 may be configured to receive the responded portion of the Activated RF range signals. In one embodiment, the Activated RF range signals may be transmitted into the food, and electromagnetic energy may be responded from the food. It can be noted that effective monitoring of the parameter levels is facilitated by an electrical response of parameters against the transmitted Activated RF range signals. Further, the electromagnetic energy responded from the food may be received by the one or more RX antennas 120. The sensor module 130 converts to digital using the AD converter 122. For example, the AD converter 122 may be configured to convert the Activated RF range signals from an analog signal into a digital processor readable format. The sensor module 130 stores the RX converted signal data in memory 124. The sensor module 130 stores the received signal from the RX antennas 120 that has been converted to a digital processor readable format in memory 124. The sensor module 130 correlates the RF signals with ground truth data to determine the parameter levels. The sensor module 130 may be configured to execute an AI correlation between the real-time ground truth data and the RX-converted data. In one embodiment, the AI correlation between the real-time ground truth data and the RX-converted data is executed to determine whether the RX-converted data corresponds to the real-time ground truth data. The ground truth data may be determined by another sensor that identifies the parameter levels when a waveform was transmitted and received. Machine learning processes may be performed to identify specific parameter waveforms, in which complex responded signals from stepped frequencies transmit signals that may be related to parameter levels. The memory 124 is used in real-time to compare received waveforms from the RX antennas 120 to a standard waveform database 126 stored in memory 124 to identify the parameter level for the received waveform from the RX antennas 120. In one embodiment, the standard waveform database 126 may be configured to store the filtered RF signal received from the one or more RX antennas 120 of the food analyzer 102. The standard waveform database 126 may store the signal waveforms for the TX antennas 118 and the received signal waveforms for the RX antennas 120. The database may include the parameter levels with the corresponding signal waveform, received waveform, and the TX antennas 118 and RX antennas 120 that were used. The standard waveform database 126 may include transmit signals and response signals for specific types of food, a specific type or parameter, and the parameter level, etc., which may enable the sensor module 130 to send a plurality of transmit signals to determine the type of food and the parameter levels for a plurality of parameters. For example, the user may activate the food analyzer 102, and the first transmit signal may be 500 MHz, and the response signal may be 425 MHz, and the transmit signal and response signal may correlate to the type of food being beef but not correlate to the food being pork, chicken, fish, etc., allowing the sensor module 130 to determine the food is beef. The sensor module 130 may then send a plurality of transmit signals to the beef to determine the various parameter levels of the beef to determine if the parameter levels are in a safe or unsafe range once compared to the integration database 134 in the process described in the integration module 132. The sensor module 130 stores the parameter levels in the sensor database 136. The sensor module 130 may store the type of food detected and the parameter levels, such as the food's pH levels, bacteria levels, levels of volatile organic compounds, enzyme levels, oxidation levels, etc., in the sensor database 136. In some embodiments, the sensor module 130 may collect parameter levels of the food by sending, receiving, converting, and then correlating the data for a first parameter and then a second parameter separately, in parallel, etc. The sensor module 130 returns to the base module 128. The base module 128 initiates, at step 204, the integration module 132. The integration module 132 begins by being initiated by the base module 128. In some embodiments, the integration module 132 may be initiated when a new data entry is stored in the sensor database 136 to determine if a corresponding notification is stored in the integration database 134. The integration module 132 extracts the data stored in the sensor database 136. The integration module 132 extracts the data, such as the detected food type, the parameter being measured, and the parameter level. The integration module 132 compares the extracted data from the sensor database 136 to the integration database 134. The integration database 134 may be a preloaded or previously created database that may contain a set of rules that, if met, a corresponding notification may be extracted and displayed on the display 110 to notify the user of the health and safety status of the food. The integration database 134 may contain a list of ranges for a plurality of food parameters in which, if the parameter levels fall in-between, above, or below the ranges, a corresponding notification may be displayed to the user. The integration database 134 may contain the type of food detected, the parameter that is being measured, the parameter range or threshold, and the corresponding notification. In some embodiments, the integration database 134 may contain safe ranges, unsafe ranges, ranges related to the time remaining until the food spoils, etc., such as pH levels, bacteria levels, volatile organic compounds, enzyme levels, oxidation, lipid content, protein content, myoglobin concentration, trimethylamine, total volatile basic nitrogen, free fatty acids, water content, residual antibiotics, and pesticides, etc., etc. Fresh meat has very low levels of bacteria, and as it spoils, bacteria like pseudomonas, lactobacillus, and yeast will rapidly multiply, and measuring these can indicate spoilage. Acceptable levels are below 105 colony-forming units per gram. Spoiling meat gives off chemicals like putrescine, cadaverine, and ammonia. These Volatile organic compounds levels increase dramatically as meat goes bad and may be measured to check freshness. Enzymes like catalase and galactosidase are produced during spoilage and measuring these enzyme levels may help determine meat freshness. Rancidity from fat oxidation causes meat to spoil and may be measured by checking for substances like peroxides, aldehydes, and ketones. The integration module 132 determines if there is a corresponding notification stored in the integration database 134. For example, if the food detected is beef or pork and the parameter is the pH level of the food, the parameter range may be 5.4-6.2 pH, and the corresponding notification may be that the beef or pork was detected and is safe to consume. If the food type detected is chicken and the parameter is the pH level of the food, the parameter range may be 5.5-6.5 pH, and the corresponding notification may be that chicken was detected and is safe to consume. Meat with pH levels within these safe ranges is considered suitable for consumption. However, if the pH level of meat falls outside these ranges, it may indicate potential safety concerns. If the food type detected is beef and the parameter is the pH level, and the beef's pH level is above 6.5, the corresponding notification may be that beef was detected and is not safe to consume. If the food type detected is chicken and the parameter is the pH level, and the chicken's pH level is below 5, the corresponding notification may be that chicken was detected and is not safe to consume. If the food type detected is pork and the parameter is the pH level, and the pork's pH level is above 6.5, the corresponding notification may be that pork was detected and is not safe to consume. A pH level higher than the safe range could indicate the presence of harmful bacteria or spoilage microorganisms and may lead to the development of undesirable flavors and textures in the meat. A pH lower than the safe range might indicate excessive acid production due to prolonged storage, spoilage, or fermentation. Highly acidic meat may have an off-putting sour taste and may not be safe for consumption. If the food type detected is chicken and the parameter is Salmonella, the parameter range may be above 1 CFU, colony-forming unit, per gram with a corresponding notification of detected chicken, salmonella warning, do not consume. Salmonella should ideally be absent or at very low levels in meat products. The safe range is generally considered to be less than 1 CFU (colony-forming unit) per gram. Dangerous levels of Salmonella in meat would be anything above the acceptable safe range, such as greater than 1 CFU per gram. High levels of Salmonella pose a significant risk of causing foodborne illness. If the food type detected is beef and the parameter is Campylobacter, the parameter range may be above 100 CFU per gram with a corresponding notification of detected beef, campylobacter warning, do not consume. Campylobacter should ideally be absent or at very low levels in meat products, with a safe range generally considered to be less than 100 CFUs per gram. Dangerous levels of Campylobacter would be above the acceptable safe range, such as greater than 100 CFUs per gram, and high levels of Campylobacter can cause foodborne illness. If the food type detected is pork and the parameter is E. coli, the parameter range may be above 1 CFU per gram with a corresponding notification of detected pork, E. coli warning, do not consume. E. coli is naturally present in the intestines of animals and humans, but certain strains can be harmful, and the safe range for pathogenic E. coli in meat is typically less than 1 CFU per gram. Dangerous levels of pathogenic E. coli would be above the acceptable safe range, such as greater than 1 CFU per gram. High levels of pathogenic E. coli can lead to severe foodborne illnesses. If it is determined that there is a corresponding notification stored in the integration database 134, the integration module 132 extracts the notification from the integration database 134. The integration module 132 extracts the notification, such as beef was detected and is safe to consume, the chicken was detected and is safe to consume, pork was detected and is safe to consume, beef was detected and is not safe to consume, chicken was detected and is not safe to consume, pork was detected and is not safe to consume, detected chicken, salmonella warning, do not consume, detected beef, campylobacter warning, do not consume, detected pork, E. coli warning, do not consume. The integration module 132 displays the extracted notification from the integration database 134 on the display 110. The integration module 132 displays the notification, such as beef was detected and is safe to consume, the chicken was detected and is safe to consume, pork was detected and is safe to consume, beef was detected and is not safe to consume, chicken was detected and is not safe to consume, pork was detected and is not safe to consume, detected chicken, salmonella warning, do not consume, detected beef, campylobacter warning, do not consume, detected pork, E. coli warning, do not consume. If it is determined that there is not a corresponding notification stored in the integration database 134 or after the notification is displayed, the integration module 132 returns to the base module 128.

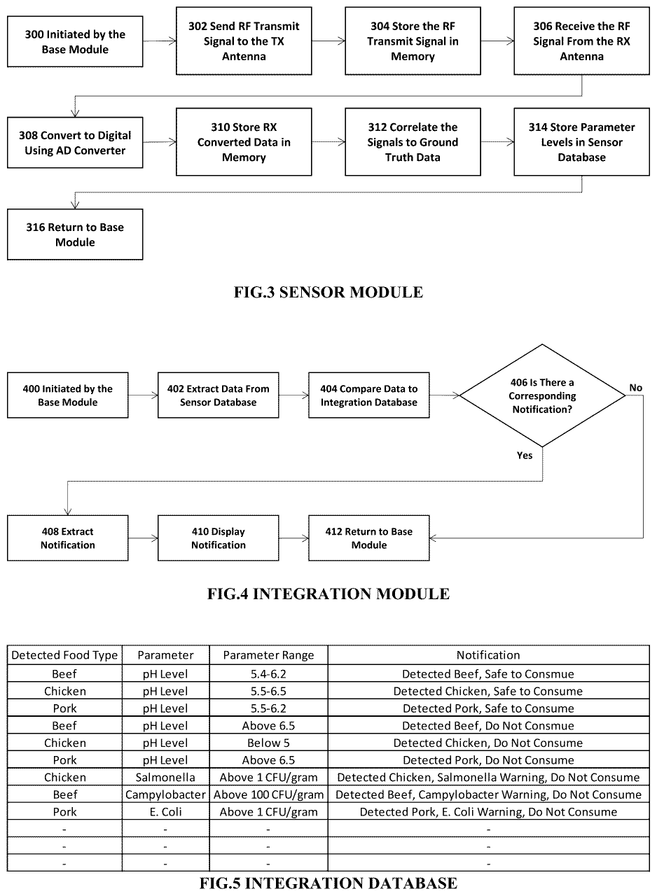

FIG. 3 illustrates an example operation of the sensor module 130. The process begins with the sensor module 130 being initiated, at step 300, by the base module 128. In some embodiments, the sensor module 130 may be initiated through the user activating the food analyzer 102 by selecting an option on the control panel 112. In some embodiments, the sensor module 130 may be initiated a plurality of times or instances to collect data on multiple parameters of the food being analyzed. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 sends, at step 302, the RF transmit signal to the TX antennas 118. The one or more TX antennas 118 may be configured to transmit the Activated RF range signals at a predefined frequency. In one embodiment, the predefined frequency may correspond to a range suitable for food. For example, the one or more TX antennas 118 transmit Activated RF range signals at a range of 500 MHz to 300 GHz. In some embodiments, the sensor module 130 may be initiated a first time to detect the type of food being analyzed by sending a plurality of transmit signals and receiving a plurality of response signals and determining if any of the RF signals correlate with a specific type of food, such as beef, chicken, pork, fish, etc. Then the sensor module 130 may be initiated a plurality of times to determine the parameter levels of the detected type of food. The sensor module 130 stores, at step 304, the RF transmit signal to memory 124. The sensor module 130 stores the transmitted signal to memory 124, such as Activated RF range signals at a range of 500 MHz to 300 GHz. The sensor module 130 receives, at step 306, the RF signal from the RX antennas 120. The one or more RX antennas 120 may be configured to receive the responded portion of the Activated RF range signals. In one embodiment, the Activated RF range signals may be transmitted into the food, and electromagnetic energy may be responded from the food. It can be noted that effective monitoring of the parameter levels is facilitated by an electrical response of parameters against the transmitted Activated RF range signals. Further, the electromagnetic energy responded from the food may be received by the one or more RX antennas 120. The sensor module 130 converts, at step 308, to digital using the AD converter 122. For example, the AD converter 122 may be configured to convert the Activated RF range signals from an analog signal into a digital processor readable format. The sensor module 130 stores, at step 310, the RX converted signal data in memory 124. The sensor module 130 stores the received signal from the RX antennas 120 that has been converted to a digital processor readable format in memory 124. The sensor module 130 correlates, at step 312, the RF signals with ground truth data to determine the parameter levels. The sensor module 130 may be configured to execute an AI correlation between the real-time ground truth data and the RX-converted data. In one embodiment, the AI correlation between the real-time ground truth data and the RX-converted data is executed to determine whether the RX-converted data corresponds to the real-time ground truth data. The ground truth data may be determined by another sensor that identifies the parameter levels when a waveform was transmitted and received. Machine learning processes may be performed to identify specific parameter waveforms, in which complex responded signals from stepped frequencies transmit signals that may be related to parameter levels. The memory 124 is used in real-time to compare received waveforms from the RX antennas 120 to a standard waveform database 126 stored in memory 124 to identify the parameter level for the received waveform from the RX antennas 120. In one embodiment, the standard waveform database 126 may be configured to store the filtered RF signal received from the one or more RX antennas 120 of the food analyzer 102. The standard waveform database 126 may store the signal waveforms for the TX antennas 118 and the received signal waveforms for the RX antennas 120. The database may include the parameter levels with the corresponding signal waveform, received waveform, and the TX antennas 118 and RX antennas 120 that were used. The standard waveform database 126 may include transmit signals and response signals for specific types of food, a specific type or parameter, and the parameter level, etc., which may enable the sensor module 130 to send a plurality of transmit signals to determine the type of food and the parameter levels for a plurality of parameters. For example, the user may activate the food analyzer 102, and the first transmit signal may be 500 MHz, and the response signal may be 425 MHz, and the transmit signal and response signal may correlate to the type of food being beef but not correlate to the food being pork, chicken, fish, etc., allowing the sensor module 130 to determine the food is beef. The sensor module 130 may then send a plurality of transmit signals to the beef to determine the various parameter levels of the beef to determine if the parameter levels are in a safe or unsafe range once compared to the integration database 134 in the process described in the integration module 132. The sensor module 130 stores, at step 314, the parameter levels in the sensor database 136. The sensor module 130 may store the type of food detected and the parameter levels, such as the food's pH levels, bacteria levels, levels of volatile organic compounds, enzyme levels, oxidation levels, etc., in the sensor database 136. In some embodiments, the sensor module 130 may collect parameter levels of the food by sending, receiving, converting, and then correlating the data for a first parameter and then a second parameter separately, in parallel, etc. The sensor module 130 returns, at step 316, to the base module 128.

FIG. 4 illustrates an example operation of the integration module 132. The process begins with the integration module 132 being initiated, at step 400, by the base module 128. In some embodiments, the integration module 132 may be initiated when a new data entry is stored in the sensor database 136 to determine if a corresponding notification is stored in the integration database 134. The integration module 132 extracts, at step 402, the data stored in the sensor database 136. The integration module 132 extracts the data, such as the detected food type, the parameter being measured, and the parameter level. The integration module 132 compares, at step 404, the extracted data from the sensor database 136 to the integration database 134. The integration database 134 may be a preloaded or previously created database that may contain a set of rules that, if met, a corresponding notification may be extracted and displayed on the display 110 to notify the user of the health and safety status of the food. The integration database 134 may contain a list of ranges for a plurality of food parameters in which, if the parameter levels fall in-between, above, or below the ranges, a corresponding notification may be displayed to the user. The integration database 134 may contain the type of food detected, the parameter that is being measured, the parameter range or threshold, and the corresponding notification. In some embodiments, the integration database 134 may contain safe ranges, unsafe ranges, ranges related to the time remaining until the food spoils, etc., such as pH levels, bacteria levels, volatile organic compounds, enzyme levels, oxidation, lipid content, protein content, myoglobin concentration, trimethylamine, total volatile basic nitrogen, free fatty acids, water content, residual antibiotics, and pesticides, etc., etc. Fresh meat has very low levels of bacteria, and as it spoils, bacteria like pseudomonas, lactobacillus, and yeast will rapidly multiply, and measuring these can indicate spoilage. Acceptable levels are below 105 colony-forming units per gram. Spoiling meat gives off chemicals like putrescine, cadaverine, and ammonia. These Volatile organic compounds levels increase dramatically as meat goes bad and may be measured to check freshness. Enzymes like catalase and galactosidase are produced during spoilage, and measuring these enzyme levels may help determine meat freshness. Rancidity from fat oxidation causes meat to spoil and may be measured by checking for substances like peroxides, aldehydes, and ketones. The integration module 132 determines, at step 406, if a corresponding notification is stored in the integration database 134. For example, if the food detected is beef or pork and the parameter is the food's pH level, the parameter range may be 5.4-6.2 pH, and the corresponding notification may be that the beef or pork was detected and is safe to consume. If the food type detected is chicken and the parameter is the pH level of the food, the parameter range may be 5.5-6.5 pH, and the corresponding notification may be that chicken was detected and is safe to consume. Meat with pH levels within these safe ranges is considered suitable for consumption. However, if the pH level of meat falls outside these ranges, it may indicate potential safety concerns. If the food type detected is beef and the parameter is the pH level, and the beef's pH level is above 6.5, the corresponding notification may be that beef was detected and is not safe to consume. If the food type detected is chicken and the parameter is the pH level, and the chicken's pH level is below 5, the corresponding notification may be that chicken was detected and is not safe to consume. If the food type detected is pork and the parameter is the pH level, and the pork's pH level is above 6.5, the corresponding notification may be that pork was detected and is not safe to consume. A pH level higher than the safe range could indicate the presence of harmful bacteria or spoilage microorganisms and may lead to the development of undesirable flavors and textures in the meat. A pH lower than the safe range might indicate excessive acid production due to prolonged storage, spoilage, or fermentation. Highly acidic meat may have an off-putting sour taste and may not be safe for consumption. If the food type detected is chicken and the parameter is Salmonella, the parameter range may be above 1 CFU, colony-forming unit, per gram with a corresponding notification of detected chicken, salmonella warning, do not consume. Salmonella should ideally be absent or at very low levels in meat products. The safe range is generally considered to be less than 1 CFU (colony-forming unit) per gram. Dangerous levels of Salmonella in meat would be anything above the acceptable safe range, such as greater than 1 CFU per gram. High levels of Salmonella pose a significant risk of causing foodborne illness. If the food type detected is beef and the parameter is Campylobacter, the parameter range may be above 100 CFU per gram with a corresponding notification of detected beef, campylobacter warning, do not consume. Campylobacter should ideally be absent or at very low levels in meat products, with a safe range generally considered to be less than 100 CFUs per gram. Dangerous levels of Campylobacter would be above the acceptable safe range, such as greater than 100 CFUs per gram, and high levels of Campylobacter can cause foodborne illness. If the food type detected is pork and the parameter is E. coli, the parameter range may be above 1 CFU per gram with a corresponding notification of detected pork, E. coli warning, do not consume. E. coli is naturally present in the intestines of animals and humans, but certain strains can be harmful, and the safe range for pathogenic E. coli in meat is typically less than 1 CFU per gram. Dangerous levels of pathogenic E. coli would be above the acceptable safe range, such as greater than 1 CFU per gram. High levels of pathogenic E. coli can lead to severe foodborne illnesses. If it is determined that there is a corresponding notification stored in the integration database 134, the integration module 132 extracts, at step 408, the notification from the integration database 134. The integration module 132 extracts the notification, such as beef was detected and is safe to consume, the chicken was detected and is safe to consume, pork was detected and is safe to consume, beef was detected and is not safe to consume, chicken was detected and is not safe to consume, pork was detected and is not safe to consume, detected chicken, salmonella warning, do not consume, detected beef, campylobacter warning, do not consume, detected pork, E. coli warning, do not consume. The integration module 132 displays, at step 410, the extracted notification from the integration database 134 on the display 110. The integration module 132 displays the notification, such as beef was detected and is safe to consume, the chicken was detected and is safe to consume, pork was detected and is safe to consume, beef was detected and is not safe to consume, chicken was detected and is not safe to consume, pork was detected and is not safe to consume, detected chicken, salmonella warning, do not consume, detected beef, campylobacter warning, do not consume, detected pork, E. coli warning, do not consume. If it is determined that there is not a corresponding notification stored in the integration database 134 or after the notification is displayed, the integration module 132 returns, at step 412, to the base module 128.

FIG. 5 illustrates an example of the integration database 134. The integration database 134 may be a preloaded or previously created database that may contain a set of rules that, if met, a corresponding notification may be extracted and displayed on the display 110 to notify the user of the health and safety status of the food. The integration database 134 may contain a list of ranges for a plurality of food parameters in which, if the parameter levels fall in-between, above, or below the ranges, a corresponding notification may be displayed to the user. The integration database 134 may contain the type of food detected, the parameter that is being measured, the parameter range or threshold, and the corresponding notification. For example, if the food detected is beef or pork and the parameter is the pH level of the food, the parameter range may be 5.4-6.2 pH, and the corresponding notification may be that the beef or pork was detected and is safe to consume. If the food type detected is chicken and the parameter is the pH level of the food, the parameter range may be 5.5-6.5 pH, and the corresponding notification may be that chicken was detected and is safe to consume. Meat with pH levels within these safe ranges is considered suitable for consumption. However, if the pH level of meat falls outside these ranges, it may indicate potential safety concerns. If the food type detected is beef and the parameter is the pH level, and the beef's pH level is above 6.5, the corresponding notification may be that beef was detected and is not safe to consume. If the food type detected is chicken and the parameter is the pH level, and the chicken's pH level is below 5, the corresponding notification may be that chicken was detected and is not safe to consume. If the food type detected is pork and the parameter is the pH level, and the pork's pH level is above 6.5, the corresponding notification may be that pork was detected and is not safe to consume. A pH level higher than the safe range could indicate the presence of harmful bacteria or spoilage microorganisms and may lead to the development of undesirable flavors and textures in the meat. A pH lower than the safe range might indicate excessive acid production due to prolonged storage, spoilage, or fermentation. Highly acidic meat may have an off-putting sour taste and may not be safe for consumption. If the food type detected is chicken and the parameter is Salmonella, the parameter range may be above 1 CFU, colony-forming unit, per gram with a corresponding notification of detected chicken, salmonella warning, do not consume. Salmonella should ideally be absent or at very low levels in meat products. The safe range is generally considered to be less than 1 CFU (colony-forming unit) per gram. Dangerous levels of Salmonella in meat would be anything above the acceptable safe range, such as greater than 1 CFU per gram. High levels of Salmonella pose a significant risk of causing foodborne illness. If the food type detected is beef and the parameter is Campylobacter, the parameter range may be above 100 CFU per gram with a corresponding notification of detected beef, campylobacter warning, do not consume. Campylobacter should ideally be absent or at very low levels in meat products, with a safe range generally considered to be less than 100 CFUs per gram. Dangerous levels of Campylobacter would be above the acceptable safe range, such as greater than 100 CFUs per gram, and high levels of Campylobacter can cause foodborne illness. If the food type detected is pork and the parameter is E. coli, the parameter range may be above 1 CFU per gram with a corresponding notification of detected pork, E. coli warning, do not consume. E. coli is naturally present in the intestines of animals and humans, but certain strains can be harmful, and the safe range for pathogenic E. coli in meat is typically less than 1 CFU per gram. Dangerous levels of pathogenic E. coli would be above the acceptable safe range, such as greater than 1 CFU per gram. High levels of pathogenic E. coli can lead to severe foodborne illnesses. In some embodiments, the integration database 134 may contain safe ranges, unsafe ranges, ranges related to the time remaining until the food spoils, etc., such as pH levels, bacteria levels, volatile organic compounds, enzyme levels, oxidation, lipid content, protein content, myoglobin concentration, trimethylamine, total volatile basic nitrogen, free fatty acids, water content, residual antibiotics, and pesticides, etc., etc. Fresh meat has very low levels of bacteria, and as it spoils, bacteria like pseudomonas, lactobacillus, and yeast will rapidly multiply, and measuring these can indicate spoilage. Acceptable levels are below 105 colony-forming units per gram. Spoiling meat gives off chemicals like putrescine, cadaverine, and ammonia. These Volatile organic compounds levels increase dramatically as meat goes bad and may be measured to check freshness. Enzymes like catalase and galactosidase are produced during spoilage, and measuring these enzyme levels may help determine meat freshness. Rancidity from fat oxidation causes meat to spoil and may be measured by checking for substances like peroxides, aldehydes, and ketones.

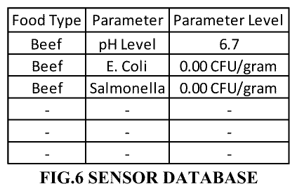

FIG. 6 illustrates an example of the sensor database 136. The sensor database 136 may be created during the process described in the sensor module 130, in which the food type and the parameter levels are stored through the data collected by the RF sensor 116. The sensor database 136 may contain the detected food type, the parameter being measured, and the parameter level. The sensor database 136 may contain a plurality of parameter readings which are compared to the ranges and thresholds stored in the integration database 134 through the process described in the integration module 132 to determine if the food being analyzed is safe for consumption or has unsafe parameter levels.

The functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

What is claimed is:

1. An integrated real-time radio frequency (RF) analysis system for food safety analysis, comprising;
an RF sensor with at least one transmit antenna that is configured to transmit a radio frequency transmit signal into food and at least one receive antenna that is configured to detect a return radio frequency signal that results from transmitting the radio frequency transmit signal into the food, and
a sensor module in communication with the RF sensor, and
an integration module in communication with the sensor module, and
an integration database in communication with the integration module, the integration database contains data on food types.
